(19) United States
(12) Patent Application Publication
(10) Pub. No.: US 2007/0191286 A1
(43) Pub. Date: Aug. 16, 2007

(54) Injectable composition

(75) Inventors: Takayuki Doen, Osaka-shi (JP); Tomoko Inoue, Osaka-shi (JP)

Correspondence Address: WENDEROTH, LIND & PONACK, L.L.P., 2033 K STREET N. W., SUITE 800, WASHINGTON, DC, 20006-1021, US

(73) Assignee: TAKEDA PHARMACEUTICAL COMPANY LIMITED, Osaka (JP)

(21) Appl. No.: 10/583,321

(22) PCT Filed: Dec. 13, 2004
(86) PCT No.: PCT/JP04/18956

§ 371 Date: Jun. 16, 2006

(30) Foreign Application Priority Data
Dec. 17, 2003 (JP) 2003-419288

(51) Int. Cl. A61K 31/44

(52) U.S. Cl. 514/23; 514/338

(57) ABSTRACT

An injectable composition comprising a combination of 2-[[[3-methyl-4-(2,2,2-trifluoroethoxy)-2-pyridinyl]methyl]sulfinyl]-1H-benzimidazole (lansoprazole), its optically active compound or a salt thereof, and a chelating agent, which is used at pH 9 to 12. The injectable composition is excellent in stability and solubility, and has such a high-quality that particulate insolubles are not formed when the composition is kept and supplied in a glass container and even in a plastic container and also when the composition is kept in these container for a long time.

### TECHNICAL FIELD

The present invention relates to an injectable composition containing a benzimidazole compound such as lansoprazole having an anti-ulcer action, and a method of its use.

### Background Art

As injectable compositions comprising a 2-[(2-pyridyl)methylsulfinyl]benzimidazole compound having an anti-ulcer action, for example, the following injectable compositions have been reported.

1) JP 2-138213 A (EP 0356143 A) discloses an injectable composition which comprises a benzimidazole compound having an anti-ulcer action and at least one of ethanol, propylene glycol and polyethylene glycol. The literature also discloses an injectable solution which contains a freeze-dried product of the benzimidazole compound dissolved in a mixture of an acidic substance and a polyethylene glycol, and further contains a saccharide such as mannitol and N-methylglucamine.

2) JP 2002-128675 A (EP 1310252 A) discloses an injectable composition using a strong alkali in a molar ratio of 1:1 relative to 2-[(2-pyridyl)methylsulfinyl]benzimidazole compound having an anti-ulcer action so that the amount of an alkali to be used is as small as possible, that a pain or a local irritation are suppressed, that the kneading operation and the complicated dissolving operation are not required, that the composition can be dissolved by a simple operation, and further that it is not necessary to attach any specific solution just for dissolving the injectable composition.

An injectable composition containing 2-[(2-pyridyl)methylsulfinyl]benzimidazole compound is used for the therapy by dissolving the composition in physiological saline or 5% glucose solution, or the like, followed by the intravenous injection. In that case, as a container for an infusion solution, nowadays, a plastic container is in a main use, though previously, a glass container was predominantly used. The plastic container includes a container made of a polyethylene, a polypropylene, etc. as a hard type, a container made of these materials as comparatively soft type and a container made of polyvinyl chloride, a container made of a copolymer of ethylene and vinyl acetate, etc. as a soft type. It is known that various plastic containers contain different additives such as a mold releasing agent, catalyst, etc., which are added when manufactured according to the manufacturer. European pharmacopoeia provides for the material of a plastic container for an injectable infusion that the concentration of the ion of metal such as aluminum, zinc, titanium, etc, eluted after 100 g of the material of a plastic container has been boiled and refluxed with hydrochloric acid for one hour is not more than 1 ppm. However, no similar provision exists in U.S.A., and it is recognized that in a part of an infusion container among such containers marketed in the world, the amount of the elution of the metal ion is large.

### DISCLOSURE OF THE INVENTION

The object of the present invention is to provide a high-quality injectable composition, comprising 2-[(2-pyridyl)methylsulfinyl]benzimidazole compound, which is more excellent in stability and solubility, and further is free from formation of particulate insolubles, even when the injectable composition is kept and supplied in a plastic container as well as in a glass container.

The present inventors have studied intensively to solve the above problems, and found that the formation of particulate insolubles from metal ions eluted from a plastic container and 2-[(2-pyridyl)methylsulfinyl]benzimidazole compound can be controlled by using edetic acid or its salt in a weight ratio of about 0.03% to about 67%, preferably about 0.3% to about 33%, more preferably about 0.6% to about 6.7% relative to the active ingredient, particularly lansoprazole, its optically active compound or a salt thereof, and that the injectable composition containing a benzimidazole compound can be filled in a plastic bag such as an infusion bag or plastic vial, kept therein and supplied therefrom. The present inventors have further studied based on the above findings and accomplished the present invention.

That is, the present invention relates to:

(1) An injectable composition comprising a combination of 2-[[[3-methyl-4-(2,2,2-trifluoroethoxy)-2-pyridinyl]methyl]sulfinyl]-1H-benzimidazole (lansoprazole), its optically active compound or a salt thereof, and a chelating agent, which is used at pH 9 to 12;

(2) The injectable composition according to the above (1), which comprises a strong alkali in an amount of about 1 to about 3 equivalent relative to one mol of lansoprazole or its optically active compound;

(3) The injectable composition according to the above (2), which further comprises N-methylglucamine;

(4) The injectable composition according to the above (3), wherein the amount of N-methylglucamine is about 0.1 mg to about 1 mg relative to 1 mg of lansoprazole, its optically active compound or a salt thereof;

(5) An injectable composition comprising a solution of lansoprazole, its optically active compound or a salt thereof and a chelating agent, which is substantially free of insolubles and filled in a container, and which is used at pH 9 to 12;

(6) The injectable composition according to the above (5), wherein lansoprazole, its optically active compound or a salt thereof, and the chelating agent are separately stored and kept, and they are mixed at the time of using the composition;

(7) The injectable composition according to the above (5), which is filled in a plastic container made of a polyethylene, a polypropylene, a copolymer of polyethylene and polypropylene, a polyvinyl chloride, a copolymer of ethylene and vinyl acetate, a copolymer of ethylene and propylene, a silicone, a polybutadiene, a thermoplastic elastomer, Teflon (Registered Trade Mark), a polyurethane, a cyclic polyolefin or a polyolefin;

(8) The injectable composition according to the above (1), wherein the chelating agent is edetic acid or its salt or a derivative thereof; phosphoric acid or its salt; or citric acid or its salt;

(9) The injectable composition according to the above (1), wherein the chelating agent is a sodium salt of edetic acid;

(10) The injectable composition according to the above (1), wherein edetic acid or its salt is contained as the chelating agent in an amount corresponding to about 0.03% to about 67% by weight relative to lansoprazole, its optically active compound or a salt thereof;

(11) The injectable composition according to the above (1), which has pH of about 10.4 to about 12.0, when it is dissolved in a physiological saline or distilled water for injection in a proportion of 5 ml thereof relative to 30 mg of lansoprazole, its optically active compound or a salt thereof;

(12) The injectable composition according to the above (1), which is a freeze-dried preparation;

(13) The injectable composition according to the above (1), which further comprises a saccharide;

(14) The injectable composition according to the above (13), wherein the saccharide is a sugar alcohol;

(15) The injectable composition according to the above (13), wherein the saccharide is mannitol;

(16) The injectable composition according to the above (13), wherein the saccharide is contained in a proportion of about 0.1 mg to about 20 mg relative to 1 mg of lansoprazole, its optically active compound or a salt thereof;

(17) The injectable composition according to the above (1), which contains about 3 mg to about 10 mg of sodium hydroxide, about 8 mg to about 24 mg of N-methylglucamine, about 50 mg to about 70 mg of mannitol and about 0.009 mg to about 20. 1 mg of disodium edetate relative to 30 mg of lansoprazole, its optically active compound or a salt thereof;

(18) An injectable composition which is prepared by adding an aqueous or a non-aqueous solvent containing edetic acid or its salt to a freeze-dried injectable preparation containing 30 mg of lansoprazole, its optically active compound or a salt thereof, about 3 mg to about 10 mg of sodium hydroxide, about 8 mg to about 24 mg of N-methylglucamine and 60 mg of mannitol;

(19) The injectable composition according to the above (1), which is for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus;

(20) A method for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp;idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus, which comprises administering the injectable composition according to the above (1) to a human being; and

(21) Use of the injectable composition according to the above (1) for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus.

As the active ingredient used in the present invention, lansoprazole, that is, 2-[[[3-methyl-4-(2,2,2-trifluoroethoxy)-2-pyridinyl]methyl]sulfinyl]-1H-benzimidazole is preferable.

The active ingredient may be an optically active compound of lansoprazole such as R-form and S-form of lansoprazole. Particularly, an optically active compound such as (R)-2-[[[3-methyl-4-(2,2,2-trifluoroethoxy)-2-pyridinyl]methyl]sulfinyl]-1H-benzimidazole is preferable. The active ingredient may also be a salt of lansoprazole or its optically active compound.

The injectable composition of the present invention is characterized in that it utilizes a combination of the above active ingredient and a chelating agent. The chelating agent may be formulated with the active ingredient and, if necessary, other ingredient(s) in a preparation. Alternatively, the chelating agent may be stored and kept separately from a preparation containing the active ingredient and these are mixed to prepare an injectable composition at the time of using the composition. As the chelating agent, for example, edetic acid, its salt, a derivative thereof, phosphoric acid, its salt, citric acid, its salt, etc. may be mentioned. These chelating agents can be used alone or in combination. Particularly, edetic acid and its salt are preferable. For example, an injectable composition which contains edetic acid or its salt in a weight ratio of about 0.03% to about 67%, preferably about 0.3% to about 33%, more preferably about 0.6% to about 6.7% relative to lansoprazole, its active compound or a salt thereof, is free from the formation of particulate insolubles even in case where the composition is filled in a plastic container, thereby permitting the provision of a high-quality injectable composition. As a preferable salt of edetic acid, there may be mentioned a salt with sodium or calcium, a combination thereof, etc. In other words, a sodium salt, a calcium salt, a salt with sodium and calcium of edetic acid (calcium disodium edetate, etc.), etc. are preferable. In particular, sodium salts of edetic acid such as are more preferable, and disodium edetate is particularly preferable. Usually, edetic acid or its salt may be used in a weight ratio of about 0.03% to about 67% relative to lansoprazole, its optically active compound or a salt thereof.

In the injectable composition of the present invention which comprises a combination of lansoprazole, its optically active compound or a salt thereof, and a chelating agent, the chelating agent forms a complex compound with a metal ion eluted from a container for an infusion solution, etc. to inhibit particulate insolubles of the metal ion eluted and lansoprazole. Therefore, the present invention includes an injectable composition comprising lansoprazole, its optically active compound or a salt thereof, and a chelating agent.

As the container for the injectable composition, various containers such as glass containers, plastic containers, etc. can be used regardless of their materials. As the plastic material for the container, a polyethylene, a polypropylene, a copolymer of polyethylene and polypropylene, a polyvinyl chloride, a copolymer of ethylene and vinyl acetate, a copolymer of ethylene and propylene, a silicone, a polybutadiene, a thermoplastic elastomer, Teflon (Registered Trade Mark), a polyurethane, a cyclic polyolefin or a polyolefin can be used.

In the injectable composition of the present invention, lansoprazole, its optically active compound or a salt thereof may be contained together with a chelating agent in the same container. Alternatively, they may be separately filled in different containers and mixed with each other at the time of using the composition. Further, lansoprazole, its optically active compound or a salt thereof is enclosed in one partition of an infusion bag whose inside is separated into two partitions, and an infusion solution is enclosed in the other partition, and the chelating agent or its salt may be enclosed in either of the two partitions. Lansoprazole, its optically active compound or a salt thereof may be formulated to a preparation in a liquid form or a preparation in a solid form such as freeze-dried injectable preparation or a powdery injectable preparation. The solid injectable preparation can be dissolved in or diluted with a solvent which substantially free from a non- aqueous solvent.

Usually, the injectable composition of the present invention can be dissolved in or diluted with a solvent which substantially free from any non-aqueous solvent (or a water-soluble organic solvent) and whose medium is substantially water by incorporating a strong alkali in addition to lansoprazole, its optically active compound or a salt thereof and a chelating agent in the injectable composition. The strong alkali is used in such an amount that the composition is used at pH about 9 to about 12, and the ratio of the strong alkali to be used is usually about 1 to about 3 equivalents relative to one mole of lansoprazole, its optically active compound or a salt thereof, though it varies depending on the kind and amount of chelating agent used.

Preferably, when lansoprazole, its optically active compound or a salt thereof, and a chelating agent are dissolved by using 5 ml of physiological saline or distilled water for injection relative to 30 mg of lansoprazole, its optically active compound or a salt thereof, the resultant solution has pH of about 9 to about 12, preferable about 10.4 to about 12.0.

The injectable composition of the present invention may further contain N-methylglucamine so as to suppress the pH lowering and to stabilize the solubility when an injectable solution is prepared. The amount of N-methylglucamine to be incorporated may be about 0. 1 mg to about 1 mg relative to 1 mg of lansoprazole, its optically active compound or a salt thereof. Further, the injectable composition may contain a saccharide (e.g. a sugar alcohol such as mannitol, etc.) so as to stabilize a shape when the composition is prepared in a solid form. The amount of the saccharide to be incorporated may be about 0.1 mg to about 20 mg relative to 1 mg of lansoprazole, its optically active compound or a salt thereof. Examples of the injectable composition containing these ingredients include a composition comprising lansoprazole, its optically active compound or a salt thereof, which can be dissolved in or diluted with a solvent substantially free from a non-aqueous solvent, and may contain about 0.1 mg to about 0.8 mg of N-methylglucamine and about 1 mg to about 10 mg of a sugar alcohol relative to about 1 mg of lansoprazole, its optically active compound or a salt thereof.

Moreover, the injectable composition preferably contains each ingredient in such a ratio as about 0.009 mg to about 20.1 mg of disodium edetate, tetrasodium edetate, calcium disodium edetate or a mixture thereof, about 8 mg to about 24 mg of N-methylglucamine, about 50 mg to about 70 mg of mannitol and about 3 mg to about 10 mg of sodium hydroxide relative to 30 mg of lansoprazole, its optically active compound or a salt thereof. In the above case, disodium edetate, tetrasodium edetate and calcium disodium edetate may be enclosed in a container different from that containing other ingredients.

Usually, the injectable composition of the present invention substantially free from a non-aqueous solvent (or aqueous organic solvent) and can be dissolved in or diluted with a solvent whose medium is substantially water. Further, the injectable composition of the present invention may be a freeze-dried preparation containing each ingredient in such a ratio as about 0.009 mg to about 20.1 mg of disodium edetate, tetrasodium edetate, calcium disodium edetate or a mixture thereof, about 8 mg to about 24 mg of N-methylglucamine, about 50 mg to about 70 mg of mannitol and about 3 mg to about 10 mg of sodium hydroxide relative to 30 mg of lansoprazole, its optically active compound or a salt thereof. In this case, disodium edetate, tetrasodium edetate and calcium disodium edetate may be enclosed in a container different from that containing other ingredients. The injectable composition can be dissolved in at least one of liquids or solvents selected from the group consisting of an infusion solution such as an water for injection (distilled water for injection), an electrolytic solution (physiological saline), a nutrient infusion, etc. and can easily be prepared into an injectable solution. As the container, a glass container and a plastic container can be used.

The present invention is useful as a method for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus, by administering the injectable composition to a human being.

Further, the present invention also discloses use of the injectable composition for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus.

Incidentally, the term “an injectable composition” as used herein means not only a final injectable solution, but also an injectable composition precursor which can be prepared into a final injectable solution with the use of a dissolving solvent upon using [for example, a liquid injectable composition (a concentrated or condensed injectable composition) or a solid injectable composition (such as a freeze-dried injectable composition)].

According to the present invention, there can be provided a high-quality injectable composition in which finely particulate insolubles are not formed when the injectable composition is kept and supplied in a glass container and even in a plastic container and also when the injectable solution prepared above is kept in these containers for a long time.

### BEST MODE FOR CARRYING OUT THE INVENTION

The injectable composition of the present invention contains lansoprazole, its optically active compound or a salt thereof and a chelating agent in a weight ratio of about 0.03% to about 67%, preferably about 0.3% to about 33%, more preferably about 0.6% to about 6.7% of the chelating agent relative to lansoprazole, its optically active compound or a salt thereof.

The salt of lansoprazole or its optically active compound preferably includes a pharmaceutically acceptable salt, for example, a salt with an inorganic base, a salt with an organic base, a salt with a basic amino acid and the like.

As the preferred examples of the salt with an inorganic base, there may be mentioned, for example, an alkali metal salt such as a sodium salt and a potassium salt; an alkaline earth metal salt such as a calcium salt and a magnesium salt; and an ammonium salt, etc.

The preferred examples of the salt with an organic base include, for example, a salt with an alkylamine (e.g., trimethylamine, triethylamine), a heterocyclic amine (e.g., pyridine, picoline), an alkanolamine (e.g., ethanolamine, diethanolamine, triethanolamine), dicyclohexylamine, N,N′-dibenzylethylenediamine or the like.

The preferred examples of the salt with a basic amino acid include, for example, a salt with arginine, lysine, ornithine or the like.

Among these salts, the alkali metal salt or the alkaline earth metal salt is preferable. In particular, the sodium salt is preferable.

Lansoprazole, its optically active compound or a salt thereof can be prepared by per se known methods, for example, the methods described in JP 61-50978 A, U.S. Pat. No. 4,628,098, JP 10-195068 A, WO 98/21201 or methods based on these methods. Incidentally, the optically active compound can be obtained by an optical resolution method (e.g., a fractional recrystallization method, a chiral column method, a diastereomer method, a method with a microorganism or an enzyme), an asymmetric oxidation method.

The chelating agent includes edetic acid, its salt, a derivative thereof, phosphoric acid, its salt, citric acid, its salt, and any agent similar thereto that is capable preparing a complex compound with a metal ion. The salt includes preferably a pharmacologically acceptable salt, for example, a salt with inorganic base such as alkali metal salt (e.g., sodium, potassium, etc.), an alkaline earth metal salt (e.g., calcium, magnesium, etc.,), ammonium salt, etc. The salt also includes a salt with an organic base, a basic amino acid, etc. In particular, a sodium salt of edetic acid is preferable.

The container of the injectable composition includes a glass container and a plastic container. As the plastic container, there may be mentioned containers made of a polyethylene, a polypropylene, a copolymer of polyethylene and polypropylene, a polyvinyl chloride, a copolymer of ethylene and vinyl acetate, a copolymer of ethylene and propylene, silicone, a polybutadiene, a thermoplastic elastomer, Teflon (Registered Trade Mark), a polyurethane, a cyclic polyolefin, a polyolefin, etc.

The injectable composition of the present invention can be produced by using lansoprazole, its optically active compound or a salt thereof and about 0.01 to about 1 equivalent/L, preferably about 0.1 to about 0.6 equivalent/L, more preferably about 0.15 to about 0.25 equivalent/L of an aqueous strong alkali solution in a ratio of about 1 to about 3 equivalent of the latter relative to 1 mol of the former and by dissolving lansoprazole, its optically active compound or a salt thereof in the aqueous strong alkali solution. Thus, the present invention also includes the injectable composition obtained by this method. In this method, the aqueous strong alkali solution may be an aqueous sodium hydroxide solution.

Thus, the injectable composition of the present invention has a preventing effect from the formation of insolubles even when the composition is kept as an injectable solution in any container and supplied after the injectable solution is prepared by adding a chelating agent. Further, in the present invention, while a strong alkali is added, the amount of the strong alkali to be used can be decreased, and the solubility of lansoprazole, its optically active compound or a salt thereof can be improved. Thus, in the present invention, a pain and a local irritation by injection is suppressed by preparing the injectable composition by using lansoprazole, its optically active compound or a salt thereof and a strong alkali in a ratio of about 1 to about 3 equivalent of the latter relative to a mol of the former without using a non-aqueous solvent (or a water-soluble organic solvent). In addition, solubility of the freeze-dried preparation in at least one liquid selected from water for injection, infusion solutions and nutrient infusions can be improved by preparing a freeze-dried preparation by using lansoprazole, its optically active compound or a salt thereof and a strong alkali in a ratio of about 1 to about 3 equivalent of the latter relative to one mol of the former without using a non-aqueous solvent (or a water-soluble organic solvent)

The injectable composition of the present invention may further contain N-methylglucamine (meglumine). The content of the “N-methylglucamine” is about 0.1 mg to about 1 mg, preferably about 0.1 to about 0.8 mg relative to 1 mg of lansoprazole, its optically active compound or a salt thereof.

The lowering of pH can be prevented by addition of N-methylglucamine because of buffer action of N-methylglucamine, thereby preventing deterioration of the quality of a preparation due to precipitation of impurities. Further, by incorporating N-methylglucamine, such a high pH can be maintained as about 9 to about 11, and further, as about 8 to about 11 can be retained depending on the concentration.

The “injectable composition” of the present invention may further contain a saccharide. As the “saccharide”, there may be mentioned, for example, a monosaccharide (e.g., glucose, galactose, ribose, xylose, mannose, maltotriose, maltotetraose, etc.), a disaccharide (e.g., sucrose, lactose, cellobiose, trehalose, maltose, etc.), a trisaccharide (e.g., raffinose, etc.), a sugar alcohol (e.g., sorbitol, inositol, mannitol, etc.), a polysaccharide (e.g., dextran, chondroitin sulfate, hyaluronic acid, dextrin sulfate, etc.) and a salt thereof (e.g., sodium chondroitin sulfate, sodium hyaluronate, etc.), a cyclic saccharide (e.g., cyclodextrin, branched cyclodextrin, etc.). Of these saccharides, a sugar alcohol is preferred. Mannitol is particularly preferred.

The amount of the “saccharide” to be added is about 0.1 to 20 mg, preferably about 0.5 to about 10 mg (e.g., about 1 to about 10 mg) relative to 1 mg of lansoprazole, its optically active compound or a salt thereof.

The injectable composition of the present invention may further contain additive(s). The “additive” includes, as a pH regulator, for example, a water-soluble inorganic acid (e.g., hydrochloric acid, sulfuric acid, carbonic acid, phosphoric acid, etc.), an alkali metal salt of a water-soluble inorganic acid (e.g., sodium chloride, potassium chloride, sodium sulfate, potassium sulfate, etc.), an alkaline earth metal salt of a water-soluble inorganic acid (e.g., calcium chloride, magnesium chloride, etc.), a water-soluble organic acid (e.g., citric acid, tartaric acid, lactic acid, succinic acid, malic acid, acetic acid, oxalic acid, benzoic acid, tannic acid, gluconic acid, fumaric acid, sorbic acid, erysorbic acid, mesylic acid, mefenamic acid, etc.), an alkali metal salt of a water-soluble organic acid (e.g., sodium citrate, sodium tartrate, etc.), an alkaline earth metal salt of a water-soluble organic acid (e.g., calcium citrate, calcium lactate, magnesium gluconate, etc.), a neutral amino acid (e.g., glycine, alanine, etc.), an acidic amino acid (e.g., aspartic acid, glutamic acid, etc.), a salt of an acidic amino acid (e.g., sodium aspartate, potassium glutamate, etc.), a salt of a basic amino acid (e.g., lysine hydrochloride, arginine hydrochloride, etc.).

Moreover, if necessary, in the “injectable composition” of the present invention, there may be employed a buffer (e.g., sodium dihydrogenphosphate, disodium hydrogenphosphate, etc.), an isotonizing agent (e.g., glucose, sodium chloride, etc.), a stabilizer (e.g., sodium hydrogensulfite, etc.), a soothing agent (e.g., glucose, benzyl alcohol, mepivacaine hydrochloride, xylocaine hydrochloride, procaine hydrochloride, carbocaine hydrochloride, etc.), a preservative (e.g., p-oxybenzoate such as methyl p-oxybenzoate and propyl p-oxybenzoate, thymelosal, chlorobutanol, benzyl alcohol, etc.).

Examples of the injectable composition of the present invention include an injectable composition comprising lansoprazole, its optically active compound or a salt thereof, a chelating agent, a strong alkali (e.g., an alkali metal hydroxide such as sodium hydroxide, etc.), N-methylglucamine and a saccharide. The preferred injectable composition includes an injectable composition comprising lansoprazole, its optically active compound or a salt thereof, sodium hydroxide, an edetate, N-methylglucamine and mannitol. In such an injectable composition, the amount of each component may be about 0.009 mg to about 20.1 mg of one of disodium edetate, tetrasodium edetate and calcium disodium edetate or a combination thereof, about 8 mg to about 24 mg of N-methylglucamine, about 50 mg to about 70 mg of a sugar alcohol (e.g., mannitol, etc.) and about 3 mg to about 10 mg of sodium hydroxide relative to 30 mg of lansoprazole, its optically active compound or a salt thereof. The edetate may be separately filled in a different container and mixed with the other components at the time of using the composition.

The injectable composition of the present invention may be in a liquid form (e.g., in the form of an aqueous injectable solution, etc.), or may be in a semi-solid form (e.g., concentrated aqueous injectable composition) or in a solid form. The preferred injectable composition of the present invention is a freeze-dried preparation (a lyophilized injectable composition). The injectable composition of the present invention also includes an injectable composition dissolved in or diluted with a dissolving liquid or a diluting liquid, when it is used. The injectable composition of the present invention is adjusted to pH about 9 to about 12, when it is used.

The injectable composition of the present invention (in particular, a freeze-dried preparation) can be dissolved in or diluted with a dissolving liquid or a diluting liquid substantially free from a non-aqueous solvent (e.g., a water-soluble organic solvent such as propylene glycol, polyethylene glycol, etc.), for example, water for injection such as distilled water for injection, an infusion solution (e.g., an electrolyte liquid such as physiological saline) to prepare the injectable solution easily. Therefore, usually, the injectable composition of the present invention is substantially free from a non-aqueous solvent (e.g., a water-soluble organic solvent such as propylene glycol and polyethylene glycol). Moreover, in such an aqueous injectable composition (injectable solution), the solubility of lansoprazole, its optically active compound or a salt thereof is not deteriorated even when the solvent is substantially water (e.g., distilled water). Further, the injectable composition of the present invention may be dissolved in a non-aqueous solution, if necessary.

Incidentally, since an aqueous solution of N-methylglucamine has a sufficient buffer capacity at pH of about 9 to about 11, the lowering of pH of a solution containing lansoprazole, its optically active compound or a salt thereof can be suppressed during the production of the injectable composition comprising lansoprazole, its optically active compound or a salt thereof and re-dissolving the injectable composition, thereby preventing the deterioration of its quality.

The injectable composition of the present invention can be produced by dissolving lansoprazole, its optically active compound or a salt thereof in an aqueous strong alkali solution (e.g., an aqueous sodium hydroxide solution, etc.), adding a chelating agent and filling the solution into a vial or an ampoule, and if necessary, lyophilizing the solution. When N-methylglucamine, a saccharide, an additive, etc. are added, the injectable composition can be obtained by dissolving lansoprazole, its optically active compound or a salt thereof, the chelating agent, N-methylglucamine, the saccharide and the additive etc. in an aqueous strong alkali solution (e.g., an aqueous sodium hydroxide solution, etc.) and filling the solution into a vial or an ampoule, and if necessary, lyophilizing the solution. The composition may be prepared by filling a chelating agent in a different container.

The most preferred concentration of the “aqueous strong alkali solution” is about 0.15 to about 0.25 equivalent/L. In other words, for example, when sodium hydroxide is employed as the strong alkali, the concentration of the “aqueous sodium hydroxide solution” is about 0.15 to about 0.25 mol/L. When a strong alkali other than sodium hydroxide is employed as the strong alkali, the injectable composition of the present invention can be also produced according to the above method.

The “dissolving” of lansoprazole, its optically active compound or a salt thereof in an aqueous strong alkali solution may be carried out by per se known methods.

The “freeze-drying (lyophilization)” may be carried out by per se known methods, and is desirably carried out by freezing a solution at a temperature of not higher than −25° C., and drying the resultant with elevating the shelf temperature to 25 to 40° C. while retaining a vacuum degree of a drying oven at a pressure of not more than about 13.3 Pa, in general.

As the “glass container (vial)”, one made of a glass usable for an injectable composition is preferred. The preferred “vial” is USP TYPE I, II, III or the like, particularly TYPE I. Moreover, such a glass vial that decreases the amount to be eluted of an alkali more than usual.

Further, a plastic vial such as a vial made from a cyclic polyolefin [e.g., CZ vial manufactured by Daikyo Seiko, Ltd.] is also employed.

The configuration and the size of the vial are not specifically limited. The capacity of the vial is preferably not more than 100 mL, more preferably not more than 40 mL, and particularly not more than 20 mL. The typical examples of vials include, for example, 17P vial, 9P vial, 5P vial, and 3.5P vial.

When an “ampoule” is used, as the glass container, one made of a glass usable for an injectable composition is preferred, and as the plastic container, one made of a polyethylene, a polypropylene, a copolymer of polyethylene and a polypropylene, a polyvinyl chloride, a copolymer of ethylene and vinyl acetate, a copolymer of ethylene and propylene, silicone, a polybutadiene, a thermoplastic elastomer, Teflon (Registered Trade Mark), a polyurethane, a cyclic polyolefin or a polyolefin can be used. The configuration and the size of the ampoule are not specifically limited. The capacity of the ampoule is preferably not more than 30 mL, more preferably not more than 20 mL, and particularly not more than 10 mL. The typical examples of ampoule include, for example, 10 P ampoule, 5 P ampoule, and 3 P ampoule.

Further the injectable composition may be in the form of a pre-filled syringe in which the injectable composition is filled in advance.

A container of the injectable composition can be coated with a packaging film. The packaging film is not specifically limited and examples thereof include those of cellophane, cellophane coated with vinylidene chloride, polyethylene, oriented polypropylene coated with vinylidene chloride, nylon, oriented nylon, oriented nylon coated with vinylidene chloride, oriented polypropylene, non-oriented polypropylene, polyester, polyester coated with vinylidene chloride, aluminum, ethylene-vinyl alcohol polymer, etc. The packaging film may be transparent or colored. Further, the packaging film may have a light screening capability and may have a capability for screening the composition from light of a specific wavelength range which promotes photo-decomposition. Preferable examples of such a film include that having a capability for screening the composition from ultraviolet light and visible light. The film material is not specifically limited and may contain an ultraviolet absorber. A light screening capability may be imparted by paper. The packaging film may also have an oxygen barrier capability and may contain an oxygen absorber. Further, the packaging film may have heat resisting properties so that it can be pasteurized or sterilized. Furthermore, the film may have fine holes so as to enhance gas permeability, wherein gas permeability may be adjusted by the film thickness or the number of holes. The film may be adhered, joined or bonded to a contained by means of heating, adhesive, etc.

In case where the injectable composition of the present invention is a freeze-dried preparation and it takes long time for the solution of the injectable composition to become transparent due to vigorous foaming of the contents upon re-dissolution, the re-dissolving time can be reduced by using a vial or an ampoule coated with a silicone. As the silicone to be used in coating, there may be mentioned, a silicone oil such as a poly(dimethylsiloxane), a poly(methylhydrogensiloxane); a varnish silicone such as a methyl varnish silicone and a methyl phenyl varnish silicone. As one example of the preferred silicone, there may be mentioned KM-740 [manufactured by Shin-Etsu Chemical Co., Ltd.].

In the case where the injectable composition of the present invention is that in an aqueous liquid form, the injectable composition can be used by pulling out a predetermined amount of the composition with an injection syringe from a vial or an ampoule. In the case where the injectable composition of the present invention is a freeze-dried preparation, the preparation is utilized by re-dissolving upon using.

As to the “solvent for re-dissolving”, it is unnecessary to employ a solution containing such a non-aqueous solvent as might exhibit a toxicity when used in a high concentration, such as polyethylene glycol, etc. Examples of the solvent for re-dissolving include water for injection (distilled water for injection), an infusion solution [an electrolyte solution (e.g., physiological saline, a Ringer's solution, etc.), a nutrition infusion solution (a carbohydrate solution, (e.g., a glucose solution such as 5% (w/v) glucose solution, etc.), an injectable solution of a protein amino acid, an injectable solution of a vitamin, etc.), a blood substitute wherein an electrolyte solution and a nutrition infusion solution (e.g., a carbohydrate solution) are combined, a fat emulsion wherein fats are emulsified, etc.], and a mixed solvent of two or more kinds thereof. To the solvent may be optionally added a pH-adjusting agent (e.g., an acidic substance, a weak-alkaline substance, etc.). In this connection, the injectable composition of the present invention may be re-dissolved in an organic solvent such as ethanol, propylene glycol and polyethylene glycol, and after dissolving in the organic solvent, the injectable composition may be further diluted with a solvent such as that exemplified with respect to the above “solvent for re-dissolving”.

The above “electrolyte solution” is a solution obtained by dissolving an electrolyte in water for injection, and includes, for example, a solution comprising one or more kinds of sodium chloride, potassium chloride, calcium chloride, sodium lactate, sodium dihydrogenphosphate, magnesium carbonate and the like, a Ringer's solution of lactic acid, a Ringer's solution of acetic acid, etc. The preferred electrolyte solution includes a solution containing sodium chloride, in particular, a physiological saline [0.9% (w/v) sodium chloride solution].

The above “carbohydrate solution” is a solution obtained by dissolving a saccharide in water for injection, and includes, for example, a solution containing one or more kinds of glucose, fructose, sorbitol, mannitol, dextran and the like. The preferred carbohydrate solution includes 5 to 70% (w/v) glucose solution, especially, 5% (w/v) glucose solution and 10% (w/v) glucose solution.

The above “injectable solution of a protein amino acid” is a solution obtained by dissolving an amino acid in water for injection, and includes, for example, a solution containing one or more kinds of glycine, aspartic acid, lysine and the like.

The above “injectable solution of a vitamin” is a solution obtained by dissolving a vitamin in water for injection, and includes, for example, a solution containing one or more kinds of vitamin B₁, vitamin C and the like.

Preferred example of “the solvent for re-dissolving” includes water for injection, physiological saline, and a glucose solution (e.g., 5% (w/v) glucose solution, etc.).

Lansoprazole, its optically active compound or a salt thereof has an excellent anti-ulcer action, gastric acid secretion-inhibiting action, mucosa-protecting action, anti-Helicobacter pylori action, etc., and is of low toxicity.

The injectable composition of the present invention is useful in mammals (e.g., human beings, non-humans such as monkeys, sheep, bovines, horses, dogs, cats, rabbits, rats, mice, etc.) for the treatment and prevention of peptic(digestive) ulcer (gastric ulcer, duodenal ulcer, stomal ulcer, acute stress ulcer); gastroesophageal reflux disease [(GERD); reflux esophagitis, gastroesophageal reflux disease not involving esophagitis (Symptomatic GERD), etc.]; gastritis; Zollinger-Ellison syndrome (which is often included in peptic ulcer); NUD (Non Ulcer Dyspepsia); gastric cancer (inclusive of gastric cancer accompanied with enhanced production of interleukin-1β due to genetic polymorphism of interleukin-1); gastric MALT lymphoma; upper gastrointestinal hemorrhage due to gastric ulcer, duodenal ulcer, acute stress ulcer and acute gastric mucosal lesion, ulcer caused by a nonsteroidal anti-inflammatory agent [inclusive of ulcer due to Aspirin (low dose for preventing heart disease)]; hyperacidity and ulcer due to postoperative stress; upper gastrointestinal hemorrhage due to invasive stress (stress from major surgery necessitating intensive management after surgery, and from cerebral vascular disorder, head trauma, multiple organ failure and extensive burn necessitating intensive treatment); gastritis atrophicans after operation of endoscopic demucosation against early gastric cancer; hyperplastic polyp; idiopathic thrombocytopenic purpura; or a disease due to Helicobacter pylori (NUD, GERD, gastritis atrophicans after operation of endoscopic demucosation against early gastric cancer, hyperplastic polyp, idiopathic thrombocytopenic purpura, iron-deficiency anemia, chronic urticaria, Raynaud's phenomenon, ischemic heart disease, migraine headache, Guillan-Barre' sydrome, etc. due to Helicobacter pylori ); asthma due to gastric acid reflux, sleep disorder due to gastric acid reflux, abdominal pain due to GERD; laryngitis; chronic obstructive pulmonary disease (COPD); obstructive apneusis; and Barrett's esophagus. Particularly, the composition is useful for the treatment of gastroesophageal reflux disease (GERD); gastric ulcer, duodenal ulcer, acute stress ulcer and acute gastric mucosal lesion, etc. each of which involves haemorrhagia which is impossible to be treated by oral administration. Further, the injectable composition of the present invention is also useful for Helicobacter pylori eradication; suppression of the above-mentioned upper gastrointestinal hemorrhage; treatment and prevention of hyperacidity and ulcer due to postoperative stress; pre-anesthetic administration etc. Particularly, the composition is useful for the treatment of gastroesophageal reflux disease (GERD); gastric ulcer, duodenal ulcer, acute stress ulcer and acute gastric mucosal lesion, etc. each of which involves haemorrhagia which is impossible to be treated by oral administration of lansoprazole, its optical active compound or a salt thereof. Further, the composition is also useful for the prevention and treatment of gastroesophageal reflux disease (GERD). The injectable composition of the present invention can be administered parenterally (e.g., drip administration, intravenous administration, intramuscular administration, subcutaneous administration) for treating or preventing these diseases. In case the injectable composition of the present invention is parenterally administered to the subject to whom oral administration cannot be applied because of hemorrhage, the injectable composition of the present invention exhibits superior effect of hemostasis by parenteral administration, and once oral administration becomes possible, such parenteral administration can be replaced by oral administration.

Lansoprazole, its optically active ingredient or a salt thereof which is the active ingredient in the injectable composition of the present invention may be used in combination with other active ingredients (e.g., one to three other active ingredients).

The “other active ingredients” include, for example, substances having an anti-Helicobacter pylori action, imidazole compounds, bismuth salts, quinolone compounds, and so forth. Of these substances, preferred are substances having an anti-Helicobacter pylori action, imidazole compounds etc. The “substances having an anti-Helicobacter pylori action” include, for example, antibiotic penicillins (e.g., amoxicillin, benzylpenicillin, piperacillin, mecillinam, etc.), antibiotic cefems (e.g., cefixime, cefaclor, etc.), antibiotic macrolides (e.g., antibiotic erythromycins such as erythromycin, clarithromycin etc.), antibiotic tetracyclines (e.g., tetracycline, minocycline, streptomycin, etc.), antibiotic aminoglycosides (e.g., gentamicin, amikacin, etc.), imipenem, and so forth. Of these substances, preferred are antibiotic penicillins, antibiotic macrolides etc. The “imidazole compounds” include, for example, metronidazole, miconazole, etc. The “bismuth salts” include, for example, bismuth acetate, bismuth citrate, etc. The “quinolone compounds” include, for example, ofloxacin, ciploxacin, etc. In particular, it is preferred for Helicobacter pylori eradication that the injectable composition of the present invention is used in combination with antibiotic penicillins (e.g., amoxicillin) and/or antibiotic erythromycins (e.g., clarithromycin).

The dose per day of Lansoprazole, its optically active ingredient or a salt thereof which is the active ingredient in the injectable composition of the present invention varies depending on severity of symptom; age, distinction of sex and weight of an administration subject; time and interval of administration; species of active ingredients, etc., and is not particularly limited. For example, the dose per day is about 0.1 to about 2 mg/kg weight, and preferably about 0.2 to about 1.5 mg/kg weight, based on lansoprazole, its optically active compound or a salt thereof which is the active ingredient, when parenterally administered as a peptic anti-ulcer agent to an adult human (60 kg). The injectable composition of the present invention is administered once a day or dividedly twice to thrice per day. The concentration of lansoprazole, its optical active compound or a salt thereof in the injectable composition to be administered is about 0.001 to about 40 mg/mL, preferably about 0.01 to about 30 mg/mL, and particularly preferably about 0.03 to about 10 mg/mL.

The injectable composition of the present invention has an excellent quality, in that the composition is free from the formation of particulate insolubles in case where the pharmaceutical composition containing lansoprazole which is a 2-[(2-pyridyl)methylsulfinyl]benzimidazole compound, its optically active compound or a salt thereof is filled, kept and supplied either in a glass container or in a plastic container.

The following examples further illustrate the present invention in detail but are not to be construed to limit the scope of the invention.

As mannitol used in the following Examples, the one that complies with the Japanese Pharmacopoeia, Fourteenth Edition, European Pharmacopoeia and USP was used.

### EXAMPLE 1

2-[[[3-Methyl-4-(2,2, 2-trifluoroethoxy)-2-pyridinyl]methyl]sulfinyl]-1H-benzimidazole (lansoprazole; hereinafter briefly referred to as Compound A) was promptly dissolved in an aqueous sodium hydroxide solution (0.2 mol/L). To the solution were added mannitol, N-methylglucamine and water for injection. After dissolution, the resultant solution was subjected to sterile filtration with a filter (0.22 μm) made from Durapore (manufactured by Nihon Millipore Ltd.). The solution thus obtained (2 mL) was filled in a 17P vial (manufactured by Daiwa Special Glass, Co., Ltd.) and freeze-dried to prepare a freeze-dried injectable preparation containing Compound A (30 mg), sodium hydroxide (3.45 mg), mannitol (60 mg) and N-methylglucamine (10 mg) (hereinafter briefly referred to as Preparation A).

Preparation A was dissolved in a dissolving liquid as shown in Table 1 (5 mL) to prepare the injectable solution having the formulation as shown in Table 2. Each 5 mL portion of the injectable solutions shown in Table 2 was diluted with physiological saline (50 mL) in a infusion container made of ethylene-propylene copolymer (0.9% Sodium Chloride Injection USP manufactured by B.Braun Medical Inc.). After dilution, the amounts of particulate insolubles were measured in accordance with the Japanese Pharmacopoeia, General Tests, Insoluble Particulate Matter Test for Injection, Method 1, Light Obscuration Particle Count Test. The results are shown in Table 3.

In a plastic container made of ethylene-propylene copolymer used in U.S.A., an increase in formation of particulates somewhat recognized in Preparation A, but the formation of the particulates was suppressed by using disodium edetate in a proportion of not less than 0.5 mg relative to 30 mg of Compound A. The number of particles was sufficiently lower as compared with the number that is regulated in the Japanese pharmacopoeia that the number of particles having a particle size of not less than 10 μm is not more than 6,000 and the number of particles having a particle size of not less than 25 μm is not more than 600 per one container. Thus, it was proved that the injectable composition of the present invention could be used in the form of a plastic container.

TABLE 1

| Dissolving liquid | 1 | | 2 | | 3 | | 4 | | 5 | |
|---|---|---|---|---|---|---|---|---|---|---|
| Disodium edetate | 0 | mg | 0.5 | mg | 1.0 | mg | 1.5 | mg | 5.0 | mg |
| Water for injection | 5 | mL | 5 | mL | 5 | mL | 5 | mL | 5 | mL |

TABLE 2

| | Formulation | | | | | | | | | |
|---|---|---|---|---|---|---|---|---|---|---|
| | 1 | | 2 | | 3 | | 4 | | 5 | |
| Compound A | 30 | mg | 30 | mg | 30 | mg | 30 | mg | 30 | mg |
| N-methylglucamine | 10 | mg | 10 | mg | 10 | mg | 10 | mg | 10 | mg |
| Mannitol | 60 | mg | 60 | mg | 60 | mg | 60 | mg | 60 | mg |
| Sodium hydroxide | 3.45 | mg | 3.45 | mg | 3.45 | mg | 3.45 | mg | 3.45 | mg |
| Disodium edetate | 0 | mg | 0.5 | mg | 1.0 | mg | 1.5 | mg | 5.0 | mg |
| Water for injection | 5 | mL | 5 | mL | 5 | mL | 5 | mL | 5 | mL |

TABLE 3

Measured value of particulate matter
(Number of particles per container)

| | Particles having a | Particles having a |
|---|---|---|
| | particle size of not | particle size of not |
| | less than 10 μm | less than 25 μm |
| Formulation 1 | 2024 | 18 |
| Formulation 2 | 139 | 4 |
| Formulation 3 | 128 | 0 |
| Formulation 4 | 191 | 4 |
| Formulation 5 | 209 | 0 |

### EXAMPLE 2

Compound A and disodium edetate were promptly dissolved in an aqueous sodium hydroxide solution (0.2 mol/L). To the solution were added mannitol, N-methylglucamine and water for injection. After dissolution, the resultant solution was subjected to sterile filtration with a filter (0.22 μm) made from Durapore (manufactured by Nihon Millipore Ltd.). The solution thus obtained (2 mL) was filled in a 17P vial (manufactured by Daiwa Special Glass, Co., Ltd.) and freeze-dried to prepare a freeze-dried injectable preparation as shown in Table 4.

The preparation shown in Table 4 was dissolved in water for injection (5 mL) to prepare an injectable preparation. The pH and the foreign insoluble matter of each injectable preparation were measured in accordance with the Japanese Pharmacopoeia, General Tests, Foreign Insoluble Matter Test for Injection. The results are shown in Table 5.

After dissolution of the preparation shown in Table 4 in water for injection (5 mL), the solution had pH about 11 and met the criteria of foreign insoluble matter provided by the Japanese Pharmacopoeia, Injection. Thus, it was proved that the injectable composition of the present invention wherein disodium edetate was added was of good quality as an injectable composition.

TABLE 4

| | Formulation | | | |
|---|---|---|---|---|
| | 1 | 2 | 3 | 4 |
| Compound A | 30 mg | 30 mg | 30 mg | 30 mg |
| N-methylglucamine | 10 mg | 10 mg | 10 mg | 10 mg |
| Mannitol | 60 mg | 60 mg | 60 mg | 60 mg |
| Sodium hydroxide | 3.70 mg | 3.77 mg | 3.82 mg | 4.11 mg |
| Disodium edetate | 1.0 mg | 1.5 mg | 1.5 mg | 1.5 mg |

TABLE 5

| | pH | Foreign Insoluble Matter |
|---|---|---|
| Formulation 1 | 10.9 | clear and free from foreign |
| | | insoluble matters that is clearly |
| | | detectable. |
| Formulation 2 | 10.9 | clear and free from foreign |
| | | insoluble matters that is clearly |
| | | detectable. |
| Formulation 3 | 11.1 | clear and free from foreign |
| | | insoluble matters that is clearly |
| | | detectable. |
| Formulation 4 | 11.3 | clear and free from foreign |
| | | insoluble matters that is clearly |
| | | detectable. |

### EXAMPLE 3

Compound A was promptly dissolved in an aqueous sodium hydroxide solution (0.2 mol/L). To the solution were added mannitol, N-methylglucamine and water for injection and the mixture was dissolved. Then, disodium edetate was dissolved in water for injection together with a small amount of sodium hydroxide. Both solutions were mixed and subjected to sterile filtration with a filter (0.22 μm) made from Durapore (manufactured by Nihon Millipore Ltd.). The solution thus obtained (2 mL) was filled in a 17P vial (manufactured by Daiwa Special Glass, Co., Ltd.) and freeze-dried to prepare a freeze-dried injectable preparation as shown in Table 6.

After the freeze-dried injectable composition shown in Table 6 was stored at 40° C. and 75% RH for 3 months, the composition was dissolved in physiological saline (5 mL) to prepare the injectable solution as shown in Table 7. Each injectable solution shown in Table 7 was diluted with physiological saline (50 mL) in an infusion container made of ethylene-propylene copolymer (0.9% Sodium Chloride Injection USP manufactured by B.Braun Medical Inc.). After dilution, the amounts of particulate insolubles were measured in accordance with the Japanese Pharmacopoeia, General Tests, Insoluble Particulate Matter Test for Injection, Method 1, Light Obscuration Particle Count Test. The results are shown in Table 8.

In a plastic container made of ethylene-propylene copolymer used in U.S.A., an increase in formation of particulates somewhat recognized in Formulation 1 (corresponding to Preparation A), but the formation of the particulates was suppressed by using disodium edetate in a proportion of not less than 1.0 mg relative to 30 mg of Compound A. The number of particles was sufficiently lower as compared with the number that is regulated in the Japanese pharmacopoeia that the number of particles having a particle size of not less than 10 μm is not more than 6,000 and the number of particles having a particle size of not less than 25 μm is not more than 600 per one container. Thus, it was proved that the injectable composition of the present invention could be used in the form of a plastic container.

TABLE 6

| | Formulation | | | | |
|---|---|---|---|---|---|
| | 1 | 2 | 3 | 4 | 5 |
| Compound A | 30 mg | 30 mg | 30 mg | 30 mg | 30 mg |
| N-methylglucamine | 10 mg | 10 mg | 10 mg | 10 mg | 10 mg |
| Mannitol | 60 mg | 60 mg | 60 mg | 60 mg | 60 mg |
| Sodium hydroxide | 3.45 mg | 3.77 mg | 3.81 mg | 4.30 mg | 6.93 mg |
| Disodium edetate | 0 mg | 1.0 mg | 1.5 mg | 3.0 mg | 15.0 mg |

TABLE 7

| | Formulation | | | | | | | | | |
|---|---|---|---|---|---|---|---|---|---|---|
| | 1 | | 2 | | 3 | | 4 | | 5 | |
| Compound A | 30 | mg | 30 | mg | 30 | mg | 30 | mg | 30 | mg |
| N-methylglucamine | 10 | mg | 10 | mg | 10 | mg | 10 | mg | 10 | mg |
| Mannitol | 60 | mg | 60 | mg | 60 | mg | 60 | mg | 60 | mg |
| Sodium hydroxide | 3.45 | mg | 3.77 | mg | 3.81 | mg | 4.30 | mg | 6.93 | mg |
| Disodium edetate | 0 | mg | 1.0 | mg | 1.5 | mg | 3.0 | mg | 15.0 | mg |
| Water for injection | 5 | mL | 5 | mL | 5 | mL | 5 | mL | 5 | mL |

TABLE 8

Measured value of particulate matter
(Number of particles per container)

| | Particles having a | Particles having a |
|---|---|---|
| | particle size of not | particle size of not |
| | less than 10 μm | less than 25 μm |
| Formulation 1 | 2704 | 71 |
| Formulation 2 | 37 | 1 |
| Formulation 3 | 130 | 2 |
| Formulation 4 | 47 | 1 |
| Formulation 5 | 70 | 0 |

### INDUSTRIAL APPLICATION

The injectable composition of the present invention, which contains lansoprazole useful as an anti-ulcer agent, its optically active compound or a salt thereof, can be provided as an injectable composition having a high-quality in that any particulate insolubles are not formed when the injectable composition is kept and supplied in a glass container and even in a plastic container.

1. An injectable composition comprising a combination of 2-[[[3-methyl-4-(2,2,2-trifluoroethoxy)-2-pyridinyl]methyl]sulfinyl]-1H-benzimidazole (lansoprazole), its optically active compound or a salt thereof, and a chelating agent, which is used at pH 9 to 12.

2. The injectable composition according to claim 1, which comprises a strong alkali in an amount of about 1 to about 3 equivalent relative to one mol of lansoprazole or its optically active compound.

3. The injectable composition according to claim 2, which further comprises N-methylglucamine.

4. The injectable composition according to claim 3, wherein the amount of N-methylglucamine is about 0.1 mg to about 1 mg relative to 1 mg of lansoprazole, its optically active compound or a salt thereof.

5. An injectable composition comprising a solution of lansoprazole, its optically active compound or a salt thereof and a chelating agent, which is substantially free of insolubles and filled in a container, and which is used at pH 9 to 12.

6. The injectable composition according to claim 5, wherein lansoprazole, its optically active compound or a salt thereof, and the chelating agent are separately stored and kept, and they are mixed at the time of using the composition.

7. The injectable composition according to claim 5, which is filled in a plastic container made of a polyethylene, a polypropylene, a copolymer of polyethylene and polypropylene, a polyvinyl chloride, a copolymer of ethylene and vinyl acetate, a copolymer of ethylene and propylene, a silicone, a polybutadiene, a thermoplastic elastomer, Teflon (Registered Trade Mark), a polyurethane, a cyclic polyolefin or a polyolefin.

8. The injectable composition according to claim 1, wherein the chelating agent is edetic acid or its salt or a derivative thereof; phosphoric acid or its salt; or citric acid or its salt.

9. The injectable composition according to claim 1, wherein the chelating agent is a sodium salt of edetic acid.

10. The injectable composition according to claim 1, wherein edetic acid or its salt is contained as the chelating agent in an amount corresponding to about 0.03% to about 67% by weight relative to lansoprazole, its optically active compound or a salt thereof.

11. The injectable composition according to claim 1, which has pH of about 10.4 to about 12.0, when it is dissolved in a physiological saline or distilled water for injection in a proportion of 5 ml thereof relative to 30 mg of lansoprazole, its optically active compound or a salt thereof.

12. The injectable composition according to claim 1, which is a freeze-dried preparation.

13. The injectable composition according to claim 1, which further comprises a saccharide.

14. The injectable composition according to claim 13, wherein the saccharide is a sugar alcohol.

15. The injectable composition according to claim 13, wherein the saccharide is mannitol.

16. The injectable composition according to claim 13, wherein the saccharide is contained in a proportion of about 0.1 mg to about 20 mg relative to 1 mg of lansoprazole, its optically active compound or a salt thereof.

17. The injectable composition according to claim 1, which contains about 3 mg to about 10 mg of sodium hydroxide, about 8 mg to about 24 mg of N-methylglucamine, about 50 mg to about 70 mg of mannitol and about 0.009 mg to about 20.1 mg of disodium edetate relative to 30 mg of lansoprazole, its optically active compound or a salt thereof.

18. An injectable composition which is prepared by adding an aqueous or a non-aqueous solvent containing edetic acid or its salt to a freeze-dried injectable preparation containing 30 mg of lansoprazole, its optically active compound or a salt thereof, about 3 mg to about 10 mg of sodium hydroxide, about 8 mg to about 24 mg of N-methylglucamine and 60 mg of mannitol.

19. The injectable composition according to claim 1, which is for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus.

20. A method for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp;idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus, which comprises administering the injectable composition according to claim 1 to a human being.

21. Use of the injectable composition according to claim 1 for preventing or treating peptic ulcer, gastroesophageal reflux disease; gastritis; Zollinger-Ellison disease syndrome; NUD (Non Ulcer Dyspepsia); gastric cancer; gastric MALT lymphoma; upper gastrointestinal hemorrhage due to gastric ulcer, duodenal ulcer, acute gastroduodenal ulcer and acute gastric mucosal lesion, ulcer caused by a nonsteroidal anti-inflammatory agent; hyperacidity and ulcer due to postoperative stress; upper gastrointestinal hemorrhage due to invasive stress; gastritis atrophicans after operation of endoscopic demucosation against early gastric cancer; hyperplastic polyp; idiopathic thrombocytopenic purpura; a disease due to Helicobacter pylori; asthma due to gastric acid reflux, sleep disorder due to gastric acid reflux; abdominal pain due to GERD; Laryngitis; chronic obstructive pulmonary disease (COPD); obstructive apneusis; and Barrett's esophagus.
